(19) United States
(12) Patent Application Publication
(10) Pub. No.: US 2007/0203475 A1
(43) Pub. Date: Aug. 30, 2007

(54) Irrigating catch and removal device

(75) Inventors: Frank J. Fischer JR., Bloomington, IN (US); Anthony D. Hammack, Bloomington, IN (US)

Correspondence Address: BRINKS HOFER GILSON & LIONE/CHICAGO/COOK, PO BOX 10395, CHICAGO, IL, 60610, US

(73) Assignee: Vance Products Incorporated, Spencer, IN (US)

(21) Appl. No.: 11/703,263

(22) Filed: Feb. 7, 2007

Related U.S. Application Data
(60) Provisional application No. 60/771,409, filed on Feb. 8, 2006.

(51) Int. Cl. A61M 25/00

(52) U.S. Cl. 604/530

(57) ABSTRACT

A retrieval device is provided for capturing and retrieving stones, calculi, and other objects from a body. The retrieval device includes an irrigation orifice to irrigate a passageway. Irrigating the passageway helps to keep the viewing area free from debris and particulate matter.

### RELATED APPLICATIONS

The present patent document claims the benefit of the filing date under 35 U.S.C. §119(e) of Provisional U.S. Patent Application Ser. No. 60/771,409, filed Feb. 8, 2006, which is hereby incorporated by reference in its entirety.

### FIELD OF THE INVENTION

This invention relates to medical retrieval devices used in minimally invasive surgery, particularly those used to retrieve objects, such as stones and calculi, from a patient.

### BACKGROUND OF THE INVENTION

Stone retrieval devices are often employed in order to remove a stone from a ureter or kidney. Removal of a stone from a ureter or kidney is a difficult procedure because the area within which the medical professional works is small and narrow. A camera is used internally in order to visually monitor the status of the procedure as the medical professional attempts to remove the stone.

Because the stone may be lodged within the patient, the medical professional breaks the stone into multiple pieces. However, because the stone fragments are able to move freely within the patient, those stone fragments may directly interfere with the camera's viewing area by moving between the camera lens and the area being viewed. Because the camera lens is no longer free from debris, the medical professional can no longer visually monitor the status of the procedure. In addition, urine may also obscure the viewing area.

Thus, should the medical professional chose to remove the stone using the prior art Entraining Biological Calculi device as seen in U.S. Patent Publication No. 2005/0192593, or similar devices, the patient may be injured if the medical professional is unable to see the area in which the procedure is being performed. Therefore, there is a need for a device which enables the medical professional to visually monitor the procedure of removing a stone after the medical professional fragmentizes the stone.

### BRIEF SUMMARY OF THE INVENTION

A retrieval device is provided. The device includes a retrieval tube made from a shape memory alloy in the form of a coil having at least two loops and having a proximal portion and a distal portion and a lumen extending throughout, wherein the retrieval tube is adapted for fitting through a passageway; and at least one irrigation orifice located on the retrieval tube.

Further, a retrieval device is provided. The device includes a retrieval tube made from a shape memory alloy in the shape of at least two coils and having a proximal portion and a distal portion and a lumen extending throughout, wherein the retrieval tube is adapted for fitting through a passageway. The device further includes an irrigation orifice located on the retrieval tube, a fitting located on the proximal portion of the retrieval tube, wherein the fitting is adapted for receiving a source of irrigation fluid, and wherein the retrieval tube is adapted for remaining movably in communication with a lumen of an access sheath.

Further, a retrieval device is provided. The device includes a retrieval tube having a proximal portion, a distal portion, and a lumen, wherein the retrieval tube is adapted for fitting through a passageway. The device further includes at least one irrigation orifice located on the retrieval tube and a portion of the retrieval device is capable of transforming into a coil.

Further, a retrieval device is provided. The device includes a retrieval tube having a proximal portion, a distal portion, and a lumen. The retrieval tube is adapted for fitting through a passageway. The retrieval device further includes an irrigation orifice located on the retrieval tube, a fitting located on the proximal portion of the retrieval tube, wherein the fitting is adapted for receiving a source of irrigation fluid.

Further, a method for retrieving an object is provided. The method includes providing a retrieval device for removal of an object, wherein the retrieval device is made from a shape memory alloy in the shape of a coil having at least two loops. The method further includes providing an access sheath having at least one lumen, inserting the retrieval device into the at least one lumen of the access sheath, inserting an endoscope into the at least one lumen of the access sheath, inserting the access sheath into a passageway, locating an object to be removed, advancing the retrieval device beyond the object to be removed, transforming a portion of the retrieval device into a coil, irrigating the passageway with a fluid from the retrieval device, and removing the object using the retrieval device.

Further, a method for retrieving an object in provided. The method includes providing a retrieval device having a lumen and at least one irrigation orifice, inserting the retrieval device into a passageway, and locating an object to be removed. The method further includes advancing the retrieval device beyond the object to be removed, irrigating the passageway with a fluid from the irrigation orifice, and moving the object using the retrieval device or fluid.

### BRIEF DESCRIPTION OF THE SEVERAL VIEWS OF THE DRAWINGS

The embodiments will be further described in connection with the attached drawing figures. Throughout the specification, like reference numerals and letters refer to like elements. It is intended that the drawings included as a part of this specification be illustrative of the embodiments and should in no way be considered as a limitation on the scope of the invention.

FIG. 1 is a side view of an embodiment of the device;

FIG. 1A depicts a foot pump for use with an embodiment of the device;

FIG. 2 is a side view of an embodiment of the device depicting a use of the device;

FIG. 3 is a side view of an embodiment of the device depicting a use of the device;

FIG. 4 is a side view of an embodiment of the device depicting a use of the device;

FIG. 4A is a blow up of a portion of FIG. 4;

FIG. 5 is a side view of an embodiment of the device depicting a use of the device;

FIG. 5A is a side view of an embodiment of the device depicting a use of the device;

FIG. 6 is a flowchart depicting a method of using an embodiment of the device;

FIG. 7 depicts an alternate embodiment of the device having a single irrigation orifice;

FIG. 8 depicts an alternate embodiment of the device having an integral irrigation orifice and basket; and

FIG. 9 depicts a use of an alternate embodiment of the device having a single irrigation orifice.

### DETAILED DESCRIPTION OF PRESENTLY PREFERRED EMBODIMENTS

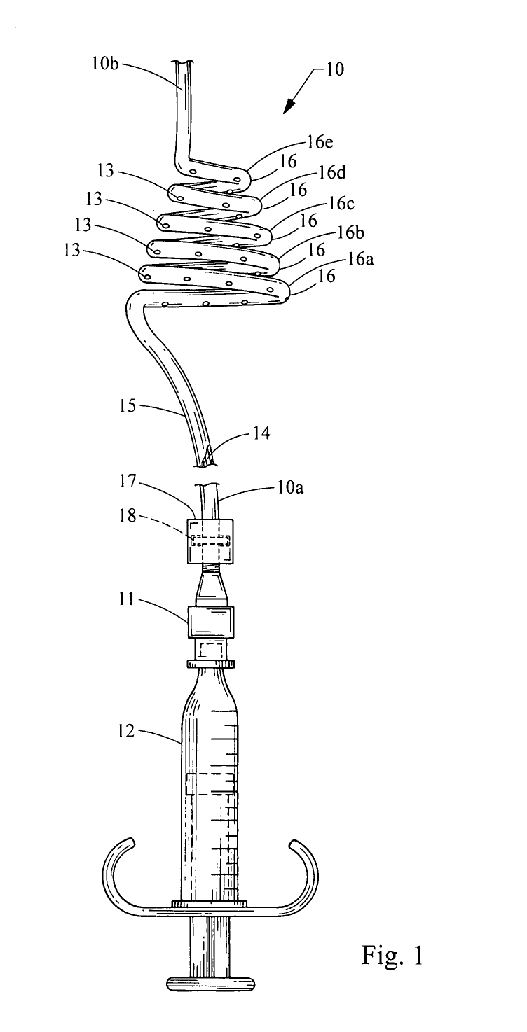

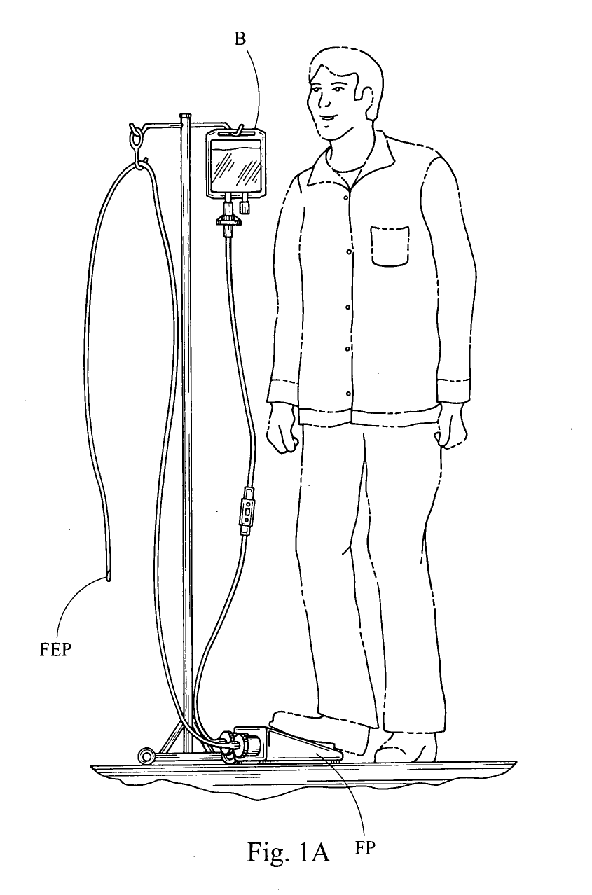

The device provides a way to capture stones, calculi, or other objects from a patient while at the same time keeping a camera's viewing area clear by irrigating the viewing area. A more detailed description of the embodiments will now be given with reference to FIGS. 1-9. The present invention is not limited to those embodiments illustrated; it specifically contemplates other embodiments not illustrated but intended to be included in the claims. The embodiments can be made from many materials, including but not limited to, Nitinol or other shape memory metals; thermoplastic materials such as polyethylene, polyurethane, fluorinated ethylene propylene, or polypropylene; and thermoset materials such as silicone, TFE, or rubber.

FIG. 1 is a side view of retrieval device 10. Retrieval device 10 consists of a retrieval tube 15 and has a proximal portion 10a and a distal portion 10b. Located at proximal portion 10a is a female Luer lock adapter (flla) 11 that is adapted for receiving syringe 12. It is contemplated that fittings other than an flla 11 may also be used. Such fittings include, but are not limited to, a Luer slip adapter. Connected to proximal portion 10A of retrieval tube 15 is connection cap 17 having o-ring 18. Flla 11 is threaded, and it screws into connection cap 17. O-ring 18 seals the connection between flla 11 and retrieval tube 15.

Syringe 12 is used for expelling an irrigation fluid into a passageway from lumen 14 of retrieval tube 15. Other sources of irrigation may also be used, including but not limited to, a bag of irrigation fluid, a foot pump, or other fluid pumping devices. FIG. 1A depicts a foot pump whereby saline (or other fluid) located in bag B is pumped out through fluid exit point FEP by pressing upon foot pump FP. Foot pumps are further disclosed in U.S. patent application Ser. No. 10/900,627 which is hereby incorporated by reference.

As shown in FIG. 1, the fluid exits retrieval device 10 through irrigation orifices/side-ports 13. Although multiple irrigation orifices 13 are depicted, it is contemplated that only one irrigation orifice or multiple irrigation orifices may be used; two to twenty-five irrigation orifices should provide a sufficient flow of irrigation fluid given a orifice diameter of 0.2540-0.5080 mm. Other diameters are contemplated.

The outer diameter of retrieval tube 15 is 7 mm-10 mm (21 Fr.-30 Fr.), however, a larger or smaller diameter is contemplated so long as the outer diameter of retrieval tube 15 is less than the diameter of the passageway through which retrieval device will enter. The inner diameter of retrieval tube 15 is 0.9652 mm; however, other diameters are contemplated. The length of retrieval tube 15 is 115 cm, however, longer or shorter lengths are also contemplated. The length of retrieval tube 15 should be long enough to reach the object to be retrieved or the place to be irrigated.

Retrieval device 10 will be thread into a passageway that contains an obstruction. A wire guide (not shown) is inserted into retrieval tube 15. The wire guide (not shown) helps to keep retrieval tube 15 relatively straight while retrieval tube 15 is positioned into a passageway and above the object to be removed. Once retrieval tube 15 is placed, the wire guide (not shown) is removed. Removing the wire guide (not shown) causes retrieval tube 15 to assume the coiled shape it was “taught” during the shape memory alloy fabrication process.

Retrieval device 10 has 5 loops 16 in the shape of a coil/corkscrew/pigtail. Having 3-8 loops is usually sufficient for capturing an object; however, having more or fewer loops is contemplated. The diameter of each succeeding loop, 16a-16e decreases such that loop 16a preferably has a 30-40% larger diameter than loop 16b, which preferably has a 30-40% larger diameter than loop 16c, which preferably has a 30-40% larger diameter than loop 16d, which preferably has a 30-40% larger diameter than loop 16e. However, the difference in loop diameter size need not be constrained to 30-40%; greater and smaller loop ratios are contemplated. Loops 16 trap the object to be removed. The sizes of loops 16a, 16b, 16c, 16d, 16e vary so that each loop is able to capture objects of differing sizes. Thus, in this embodiment, loop 16e is the smallest and thus capable of capturing an object smaller in size than loop 16a; however, other loop arrangements are contemplated.

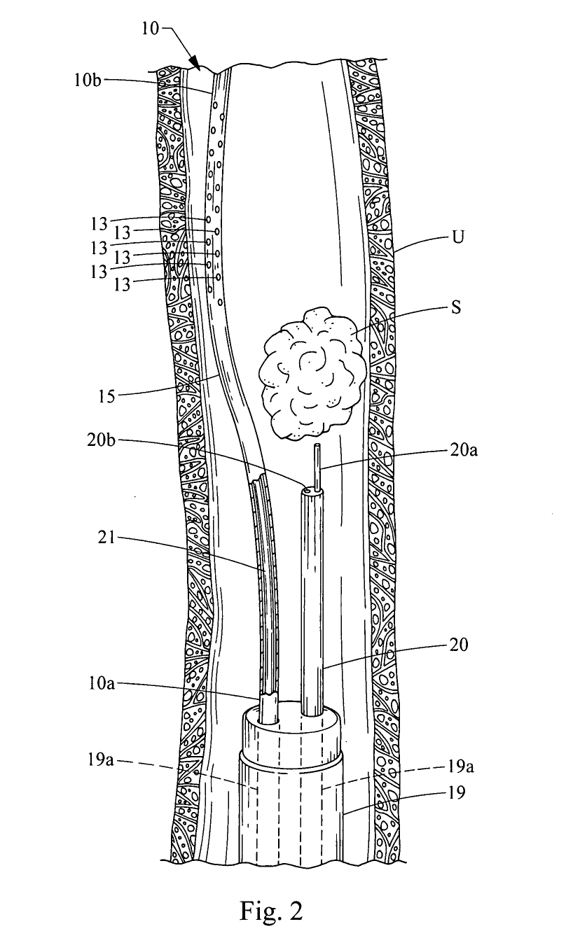

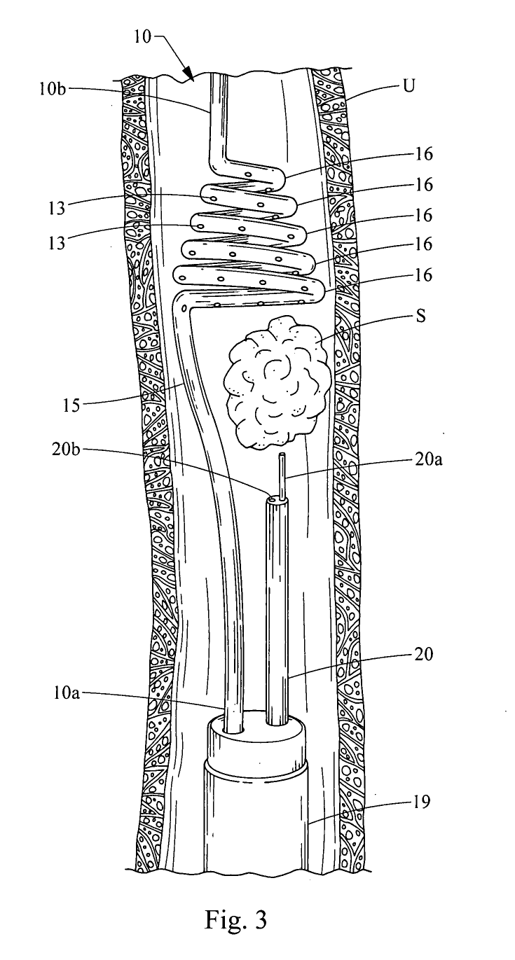

FIGS. 2 and 3 show the use of a retrieval device. FIG. 2 depicts retrieval device 10, having a proximal portion 10a and a distal portion 10b, placed in an access sheath 19 which is placed in ureter U. Access sheath 19 is shown having double-lumens 19a, however a single-lumen access sheath or an access sheath having more than two lumens will also suffice. Although shown using an access sheath, use of such a device is not required.

In FIG. 2, retrieval device 10 is thread through one of the double-lumens 19a of access sheath 19. Wire guide 21 is placed inside retrieval tube 15 to help keep retrieval tube 15 relatively straight while a portion of retrieval tube 15 having irrigation orifices 13 is placed above the location of stone S. Flexible ureteroscope 20 having camera 20b and laser 20a are positioned such that they are located below the position of stone S. Retrieval device 10 is not limited to use with ureteroscope 20; other types of devices and endoscopes can also be used. Similarly, retrieval device 10 can also be used by itself. Once retrieval device 10 is properly positioned, wire guide 21 is removed.

FIG. 3 depicts retrieval device 10 after wire guide 21, depicted in FIG. 2, is removed. Retrieval tube 15 forms a coil having loops 16. Loops 16 capture pieces of stone S.

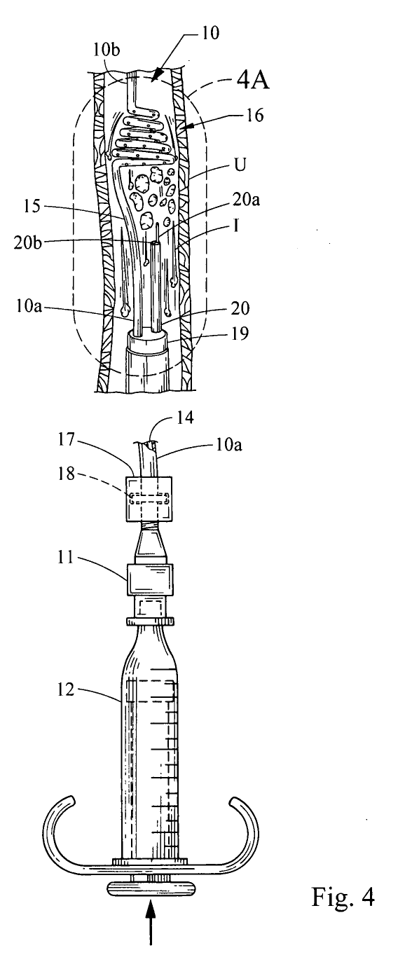

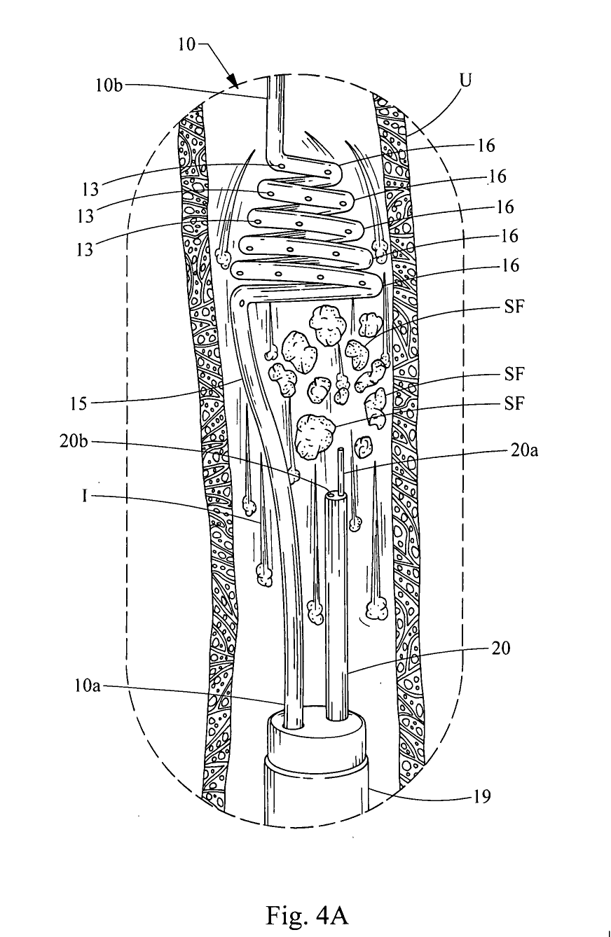

FIGS. 4 and 4A depict retrieval device 10 as it is being used to capture stone fragments SF. FIG. 4A is a blow-up of the upper-portion of FIG. 4. Camera 20b is used to help visualize the object to be retrieved, here a stone. Although camera 20b is shown being used, other visualization systems are contemplated. Laser 20a is used to break apart stone into stone fragments SF. Although laser 20a is shown being used, other object fragmentizing systems are contemplated.

Laser 20a breaks apart stone into stone fragments SF. Stone fragments SF, along with urine, may block the view of camera 20b. Blocking the view of camera 20b may result in patient injury because the medical professional is unable to view the status of the procedure. To keep the viewing area clean from stone debris and urine, irrigation fluid I is injected into retrieval tube 15 using syringe 12. Irrigation may also be used before, during, and after laser 20a is used. It is preferred, but not required, that irrigation fluid I be used throughout the procedure in order to keep the viewing area clear of debris and urine.

Irrigation fluid I exits through irrigation orifices 13 and washes away stone fragments SF and urine that block the viewing area of camera 20b. A sufficient amount of irrigation fluid I to keep the viewing area clean is generally 50 ml/min; however, more or less irrigation fluid may be used. Irrigation fluid I can consist of fluids including, but not limited to, water, saline, and contrast medium.

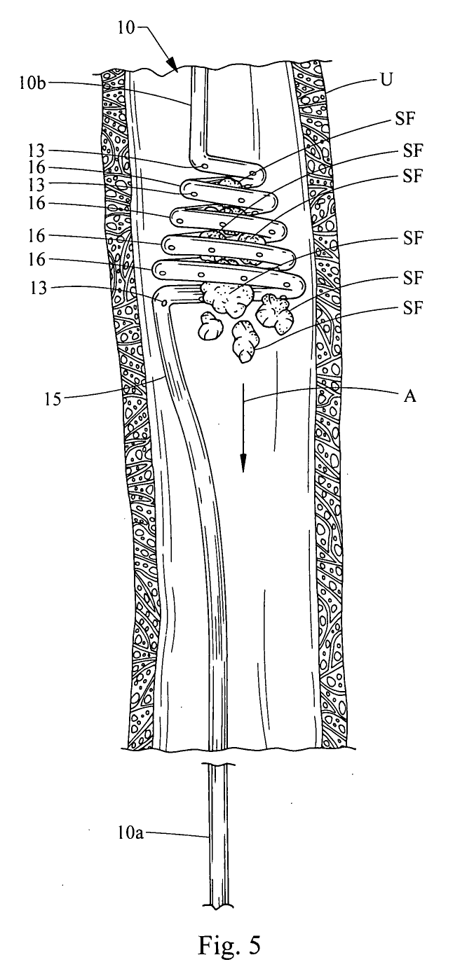

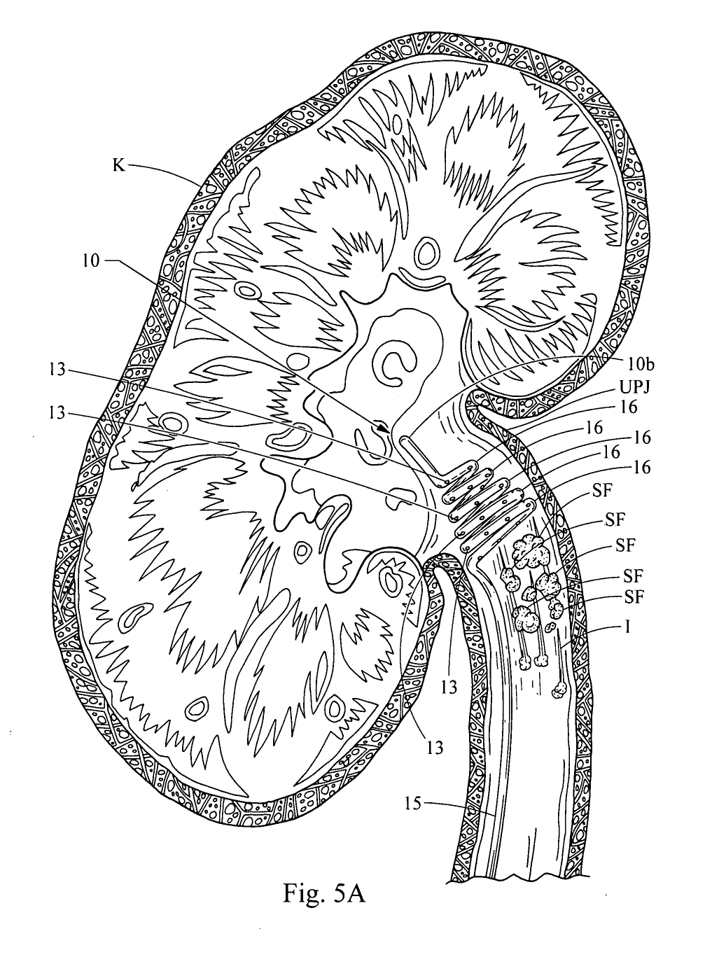

FIG. 5 depicts retrieval device 10 removing stone fragments SF using a sweeping motion in the direction of arrow A. Irrigation of the passageway may continue throughout this process. The medical professional retracts retrieval device 10 at the proximal portion 10a, by pulling or sweeping the device. As retrieval device 10 is pulled in the proximal direction, stone fragments SF are collected by loops 16, thus, allowing the medical professional to remove the object from the patient. Additionally, irrigation fluid can be dispensed through retrieval device 10 at a sufficiently high pressure so as to not only remove debris from the viewing area but to also force the debris away from the kidney and down the ureter or other bodily lumen through which the debris should travel. Thus, it may not be necessary to trap the debris using loops 16 if irrigation fluid I travels from irrigation orifices 13 having a sufficient pressure to move debris through a bodily lumen towards the exit way. A sufficient irrigation fluid pressure to move stone fragments is about 20 mmHg however, higher pressures are contemplated depending on the debris to be cleared and the area in which irrigation fluid I is being flushed.

Although retrieval device 10 is shown placed in ureter U, retrieval device 10 is not limited to such placement. For example, FIG. 5A depicts retrieval device 10 prior to capturing stone fragments SF where retrieval device 10 is positioned above the ureteropelvic junction (UPJ) near kidney K.

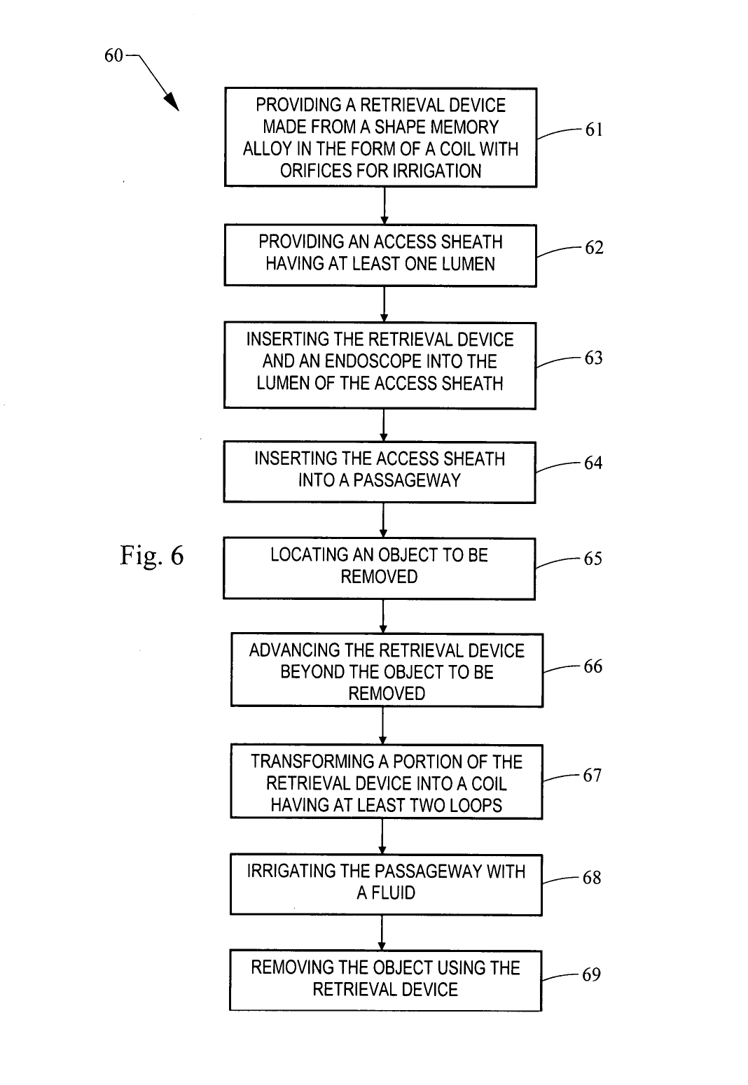

FIG. 6 depicts a method of using an embodiment of the device 60. A retrieval device made from a shape memory alloy in the form of a coil with orifices for irrigation is provided 61. As discussed above, retrieval device may have one or more irrigation orifices. Also provided is an access sheath having at least one lumen 62. The retrieval device and an endoscope are inserted into the lumen of the access sheath 63, the access sheath is inserted into a passageway 64, and the object to be removed is located 65. The object to be removed can be located by a camera that is part of the endoscope. The retrieval device is advanced beyond the object to be removed 66, and a portion of the retrieval device is transformed into a coil having at least two loops 67. The passageway is irrigated with a fluid from the retrieval device 68 to remove obstructions from the viewing area of the camera as the object to be removed is broken apart 69. The object to be removed is removed out from the passageway using the retrieval device 70. Other methods may also use a separate visualization system. The access sheath may be a Flexor® access sheath by Cook Urological Incorporated. Moreover, as described above, the irrigation fluid can be dispensed through the retrieval device with a sufficient pressure so as to not only remove debris from the viewing area but to also force the debris away from the kidney and down the ureter or other bodily lumen through which the debris should travel. Thus, it may not be necessary to trap the debris using the retrieval device if the irrigation fluid forces the debris through a bodily lumen exit way.

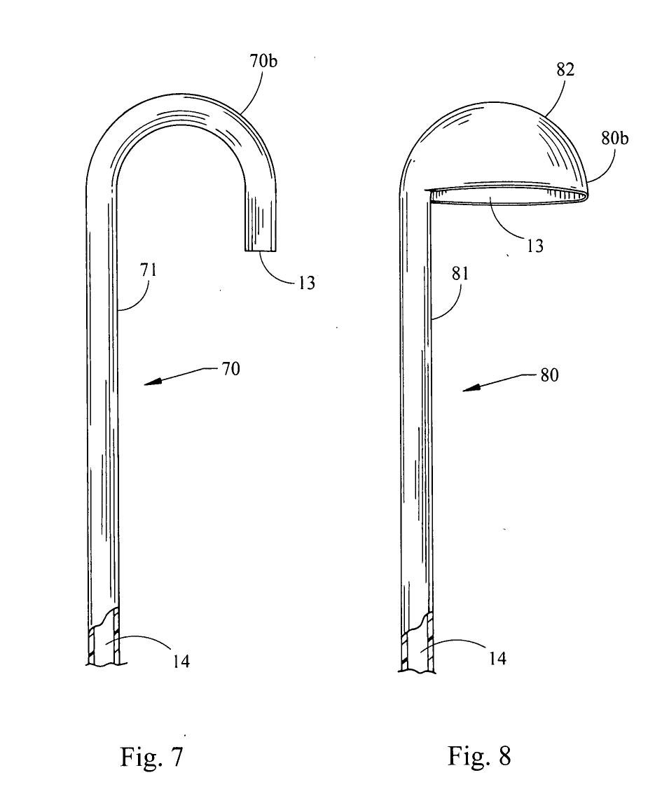

FIG. 7 depicts an alternate embodiment of a retrieval device. Retrieval device 70, having lumen 14, has a single irrigation orifice 13 that has an inner diameter similar to that of elongated tube 71. As is evident, retrieval device 70 lacks the loops of previous embodiments. Thus, it is contemplated that irrigation fluid would travel out from irrigation orifice 13 located at distal portion 70b of retrieval device 70. A sufficient pressure of irrigation fluid would be dispensed through irrigation orifice 13 so as to push debris and urine away from the camera viewing area. Retrieval device 70 is placed above the object to be retrieved in order to push the object to be retrieved towards the exit way using irrigation fluid.

FIG. 8 depicts another alternate embodiment of a retrieval device. Retrieval device 80 has an integral basket 82 and irrigation orifice 13 located at distal portion 80b of elongated tube 81. From irrigation orifice 13, an irrigation fluid could flow to clear the viewing area from debris and urine. Basket 82, like loops described above, enables capturing of debris. Additionally, it is contemplated that a sufficient pressure of irrigation fluid could be dispensed through irrigation orifice 13 so as to push debris towards exit way without needing to capture debris using basket 82.

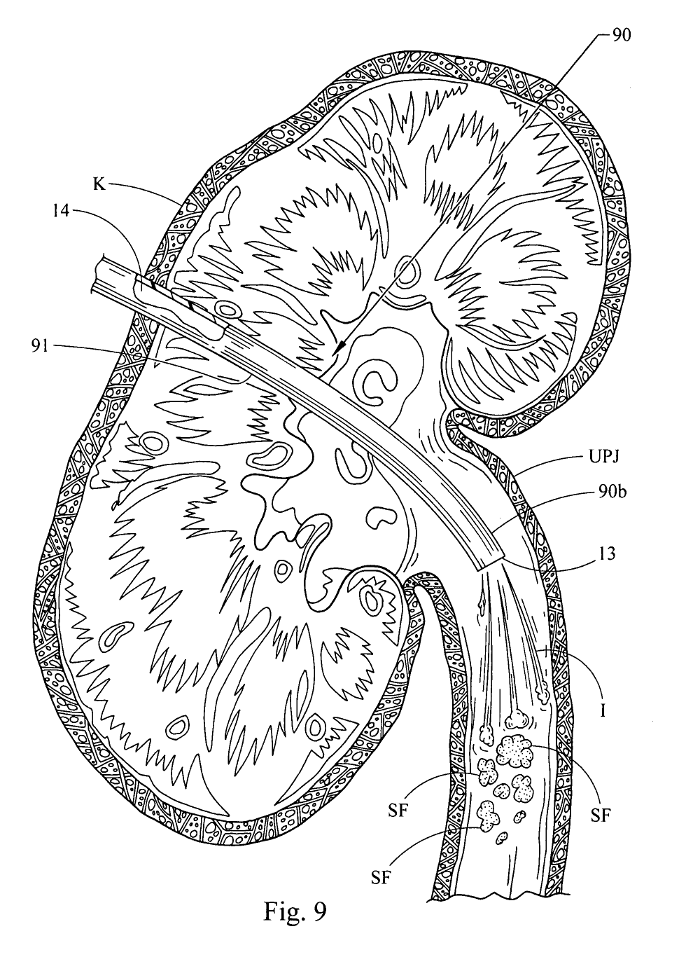

FIG. 9 depicts an alternate embodiment of a retrieval device. Retrieval device 90 has one irrigation orifice 13 located at distal portion 90b and lumen 14 extending throughout elongated tube 91. Retrieval device 90 is inserted above the object to be removed. Here, retrieval device 90 is shown inserted through kidney K and into the UPJ. Once placed above the object to be removed, in this case stone fragments SF, pressurized irrigation fluid I is expelled through irrigation orifice 13 to push and direct stone fragments SF towards the exit way where they can be retrieved.

As is evident, the embodiments provide a very effective solution for removal of stones, calculi, or other objects from a patient by a device that allows for both irrigation of a passageway and collection of objects from a body. Use of retrieval device is not limited to human patients; the device can also work in animals as well as any place where irrigation may aid in the collection and removal of objects.

The foregoing description and drawings are provided for illustrative purposes only and are not intended to limit the scope of the invention described herein or with regard to the details of its construction and manner of operation. It will be evident to one skilled in the art that modifications and variations may be made without departing from the spirit and scope of the invention. Changes in form and in the proportion of parts, as well as the substitution of equivalents, are contemplated as circumstances may suggest and render expedience; although specific terms have been employed, they are intended in a generic and descriptive sense only and not for the purpose of limiting the scope of the invention set forth in the following claims.

1. A retrieval device comprising:
a retrieval tube having a proximal portion, a distal portion, and a lumen, wherein the retrieval tube is adapted for fitting through a passageway;
at least one irrigation orifice located on the retrieval tube; and
wherein a portion of the retrieval device is capable of transforming into a coil.

2. The retrieval device of claim 1 wherein the at least one irrigation orifice further consists of at least ten irrigation orifices.

3. The retrieval device of claim 1 wherein the retrieval tube further comprises a shape memory alloy.

4. The retrieval device of claim 1 wherein the shape of the coil comprises three to eight coils.

5. The retrieval device of claim 4 wherein a diameter of the coil becomes progressively 30-40% smaller from one coil to another coil.

6. The retrieval device of claim 1 wherein the retrieval tube further comprises a fitting attached to the proximal portion.

7. The retrieval device of claim 6 wherein the fitting is adapted for receiving a source of irrigation fluid.

8. The retrieval device of claim 7 wherein the source of irrigation fluid is selected from the group consisting of a syringe, a bag, a pump, and a foot pump.

9. The retrieval device of claim 6 wherein the fitting is selected from the group consisting of a female Luer Lock adapter and a Luer slip adapter.

10. The retrieval device of claim 1 wherein the irrigation orifice has a diameter of 0.2540-0.5080 mm.

11. A retrieval device comprising:
a retrieval tube having a proximal portion, a distal portion, and a lumen, wherein the retrieval tube is adapted for fitting through a passageway;
an irrigation orifice located on the retrieval tube;
a fitting located on the proximal portion of the retrieval tube, wherein the fitting is adapted for receiving a source of irrigation fluid.

12. The retrieval device of claim 11 wherein a portion of the retrieval device is capable of transforming into a coil.

13. The retrieval device of claim 11 wherein the retrieval device further comprises a basket.

14. A method for retrieving an object, the method comprising:
providing a retrieval device having a lumen and at least one irrigation orifice;
inserting the retrieval device into a passageway;
locating an object to be removed;
advancing the retrieval device beyond the object to be removed;
irrigating the passageway with a fluid from the irrigation orifice; and
moving the object using the retrieval device or fluid.

15. The method of claim 14 wherein a portion of the retrieval device is capable of transforming into a coil.

16. The method of claim 15 further comprising irrigating the passageway with a fluid to push the object towards an exit way.

17. The method of claim 14 further comprising breaking at least a portion of the object to be removed using an object fragmentizing system.

18. The method of claim 17 wherein the fragmentizing system is a laser.

19. The method of claim 14 wherein the fluid is selected from the group consisting of water, saline, and contrast medium.

20. The method of claim 14 wherein 50 ml/min of the fluid is used for irrigating the passageway.
